(19) United States
(12) Patent Application Publication
(10) Pub. No.: US 2022/0338569 A1
(43) Pub. Date: Oct. 27, 2022

(54) Facemask

(71) Applicant: NOH PYUNG MYUNG, RIVERSIDE, CA (US)

(72) Inventor: NOH PYUNG MYUNG, RIVERSIDE, CA (US)

(21) Appl. No.: 17/725,396

(22) Filed: Apr. 20, 2022

Related U.S. Application Data
(60) Provisional application No. 63/179,115, filed on Apr. 23, 2021.

(51) Int. Cl. A41D 13/11

(52) U.S. Cl. CPC A41D 13/11

(57) ABSTRACT

A facemask has an outer surface, an inner surface, and a mouthpiece. The inner surface has either a receiver or an insert, and the mouthpiece has either an insert or a receiver that mates with the receiver or the insert of the inner surface. When the receiver and the insert are mated (or coupled), the user can wear the facemask by having the mouthpiece inside the user's mouth.

This application claims priority of a U.S. provisional patent application 63/179,115 (the “'115 Application”) with the filing date of Apr. 23, 2021. The entire content of the '115 Application is hereby incorporated by reference.

### SUMMARY

A facemask is used to prevent the transmission of airborne diseases. A commonly used facemask has a rectangular-shaped mask surface with two strings for hooking around the ears of the user. A challenge of such a facemask is that extended wear causes bruises, blisters, tenderness, and pain behind the ears. Therefore, there is a need for a facemask that does not use strings to loop around the ears of the user.

A facemask presented has an outer surface, an inner surface, and a mouthpiece. The inner surface has either a receiver or an insert, and the mouthpiece has either an insert or a receiver that mates with the receiver or the insert of the inner surface. When the receiver and the insert are mated (or coupled), the user can wear the facemask by having the mouthpiece inside the user's mouth.

The mouthpiece has a front wall and a rear wall forming a channel with a bottom, so the user wears the facemask by inserting a row of teeth in between the front wall and the rear wall of the mouthpiece. The bottom of the channel is extended beyond the front wall, forming a liprest (that is, rest for a lip) for the lip of the user. At the end (at the extremity) of the liprest has either the receiver or the insert attached to the mouthpiece. The applicant believes a preferred location of the receiver or the insert attached to the inner surface is at about the center section of the inner surface. The size of either or both the receiver and the insert may vary in length for the comfort of the user when the facemask is worn. One variation of the facemask may have the receiver or the insert of the inner surface be one inch or more in length. Another variation of the facemask for either the receiver or the insert of the inner surface is less than one inch in length.

Another variation of the facemask has the receiver having a shape of a “C” with a groove and the insert having a shape that tightly fits into the groove of the receiver. Another variation of the facemask has the receiver having a shape of a rectangular box with a hole and the insert having a shape that fits tightly into the hole of the receiver.

Another variation of the facemask has a bridge attached to the upper section of the facemask, and the facemask also has a cutout at the upper section to accommodate a portion of a nose of a user. An improvement of the facemask has the bridge having one or more bridge-adhesive sections to better secure the bridge and the upper section of the facemask on the user. The bridge-adhesive sections are located at either or both sides of the bridge over the cutout.

The cutout may be designed to cover the nose partially, substantially, all but the tip of the nose, or none of the nose except to fit snuggly below the nostrils. Also, as an improvement, the facemask may have a left flap attached to the left side of the cutout and a right flap attached to the right side of the cutout so that the left flap and the right flap cover one or more portions of the nose of the user. The left flap and the right flap may be used to prevent or reduce the air leaking from under the facemask. An improvement may be made by having the left flap and the right flap have adhesives on them to adhere to the portions of the nose to better prevent air from escaping and to better prevent fogging of any eyeglasses worn by the user.

Another improvement of the facemask is the facemask having an air vent. The air vent may be attached to a right section, a left section, a lower section, or a mouth section of the facemask. Also, to make the facemask tightly fitting and to reduce air escaping from the inside of the facemask, the facemask may have a left-adhesive section on the left section of the facemask, a right-adhesive section on the right section of the facemask, and even a lower-adhesive section on the lower section of the facemask to better secure the facemask on the user.

These and other features, aspects, and advantages of the improved facemask will become better understood with reference to the accompanying specification, writing, drawings, and submission, all of which are incorporated herein.

### BRIEF DESCRIPTION OF THE DRAWINGS

These and other features, aspects, and advantages of the improved facemask will become better understood with reference to the accompanying drawings, wherein:

FIG. 1 is an inside view of the facemask,

FIG. 2 is an outside view of the facemask,

FIG. 3A is a side view of the facemask,

FIG. 3B is a view of a mouthpiece,

FIG. 4A is a view of an insert,

FIG. 4B is a view of a receiver,

FIG. 5A is a view of the facemask showing a variation of a receiver,

FIG. 5B is a view of a variation of the mouthpiece,

FIG. 6 is a view of a row of teeth about to be placed into the mouthpiece and a variation of a receiver,

FIG. 7 is a view of a variation of the mouthpiece with an insert and a receiver for the facemask,

FIG. 8 is an inside view of a variation of the facemask,

FIG. 9 is a view of a user wearing the facemask,

FIG. 10 is a view of a left flap and a right flap of the facemask, and

FIG. 11 is a view of the user holding the mouthpiece by biting into the mouthpiece.

### DETAILED DESCRIPTION EMBODIMENTS OF THE INVENTION

A facemask is described in detail, as shown in Figs.

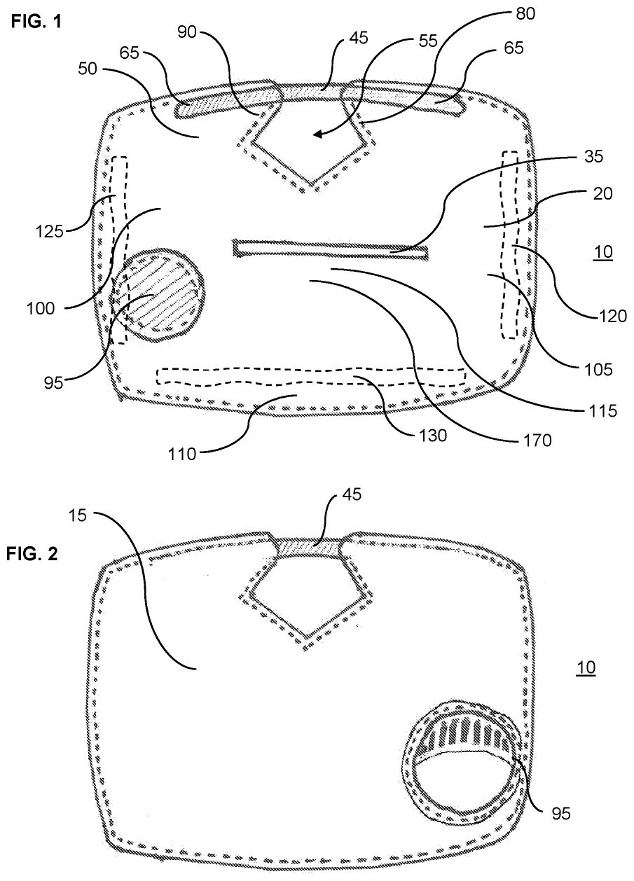

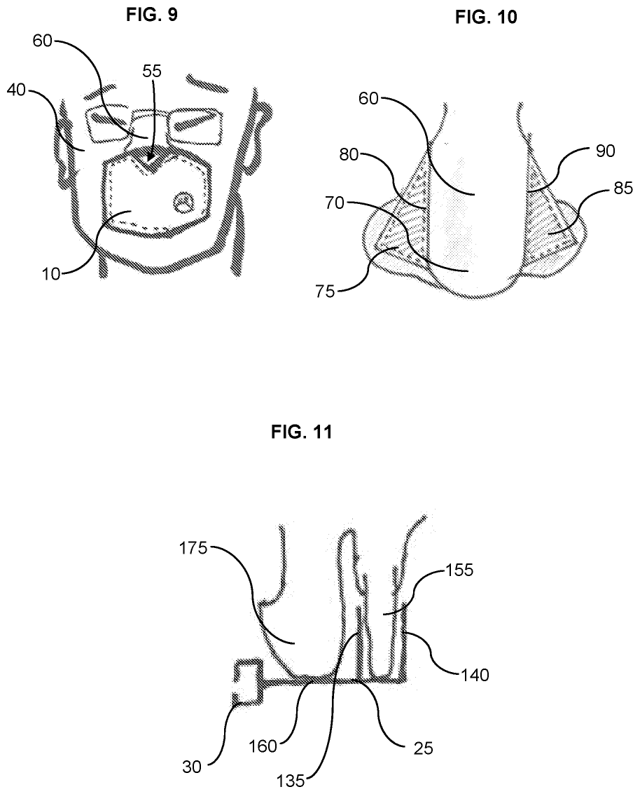

FIG. 1 and FIG. 2 show an inside view of the facemask and an outside view of the facemask 10, respectively. The facemask 10 presented has an outer surface 15, an inner surface 20, and a mouthpiece 25. The inner surface 20 has either a receiver 30 or an insert 35, and the mouthpiece 25 has either an insert 35 or a receiver 30 that mates with the receiver 30 or the insert 35 of the inner surface 20. When the receiver 30 and the insert 35 are mated (coupled or fitted together, or inserted into), the user 40 (FIG. 9) can wear the facemask 10 by having the mouthpiece 25 inside the user's mouth.

FIG. 1 and FIG. 2 also show a bridge 45 attached to the upper section 50 of the facemask 10, and the facemask 10 also has a cutout 55 at the upper section 50 to accommodate a portion of a nose 60 (FIGS. 9 and 10) of the user. An improvement of the facemask has the bridge 45 having one or more bridge-adhesive sections 65 to better secure the bridge 45 and the upper section 50 of the facemask 10 on the user's face. The bridge-adhesive sections 65 are located at either or both sides of the bridge 45 over the cutout 55.

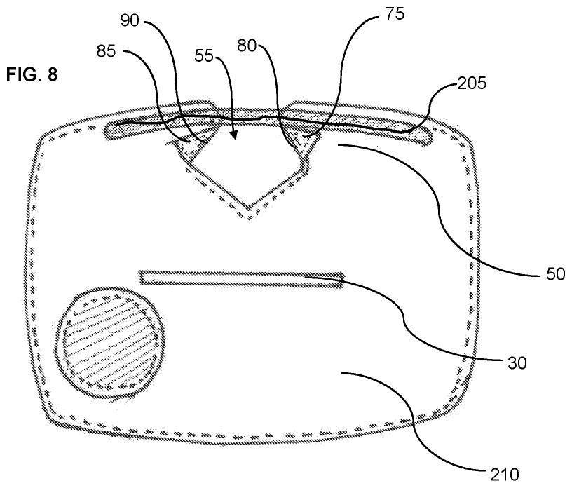

The cutout 55 may be designed to cover the nose 60 partially, substantially, all but the tip 70 of the nose 60, or none of the nose 60 except to fit snuggly below the nostrils. Also, as an improvement, the facemask 10 may have a left flap 75 (FIG. 8) attached to the left side 80 of the cutout and a right flap 85 (FIG. 8) attached to the right side 90 of the cutout so that the left flap 75 and the right flap 85 cover one or more portions of the nose of the user. The left flap 75 and the right flap 85 may be used to prevent or reduce the air leaking from under the facemask 10. An improvement may be made by having the left flap 75 and the right flap 85 have adhesives on them to adhere to the portions of the nose to better prevent air from escaping and to better prevent fogging of any eyeglasses worn by the user.

Another improvement of the facemask 10 is the facemask 10 having an air vent 95. The air vent 95 may be attached to a right section 100, a left section 105, a lower section 110, or a mouth section 115 of the facemask 10. The air vent 95 may have a replaceable filter, a charcoal-filled filter, a deodorant filter, or a chemical filter

Also, to make the facemask 10 tightly fitting and to reduce air escaping from the inside of the facemask 10, the facemask 10 may have a left-adhesive section 120 on the left section 105 of the facemask, a right-adhesive section 125 on the right section 100 of the facemask, and even a lower-adhesive section 130 on the lower section 110 of the facemask 10 to better secure the facemask 10 on the user. The adhesive used in adhesive sections 65, 120, 125, 130 should be light adhesives that can easily be removed from the face, such as having temporary adhesive strength and easily washed off.

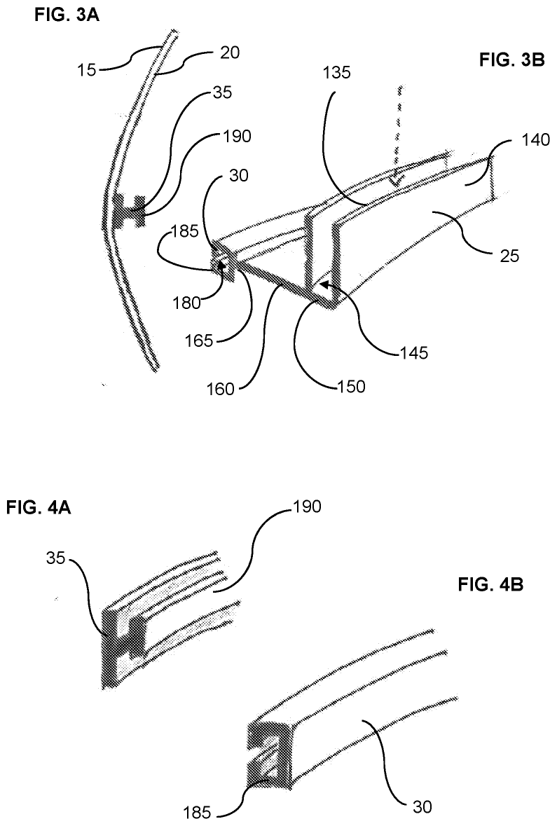

FIG. 3A shows the facemask 10 with the insert 25 on the inner surface of the facemask 10. FIG. 3B shows a mouthpiece 25. The mouthpiece 25 has a front wall 135 and a rear wall 140 forming a channel 145 with a bottom 150, so the user wears the facemask 10 by inserting a row of teeth 155 in between the front wall 135 and the rear wall 140 of the mouthpiece 10. The bottom 150 of the channel 145 is extended beyond the front wall 135, forming a liprest 160 (that is, rest for a lip) for the lip of the user. FIG. 11 shows a view of the user holding the mouthpiece 25 by biting into the mouthpiece 25.

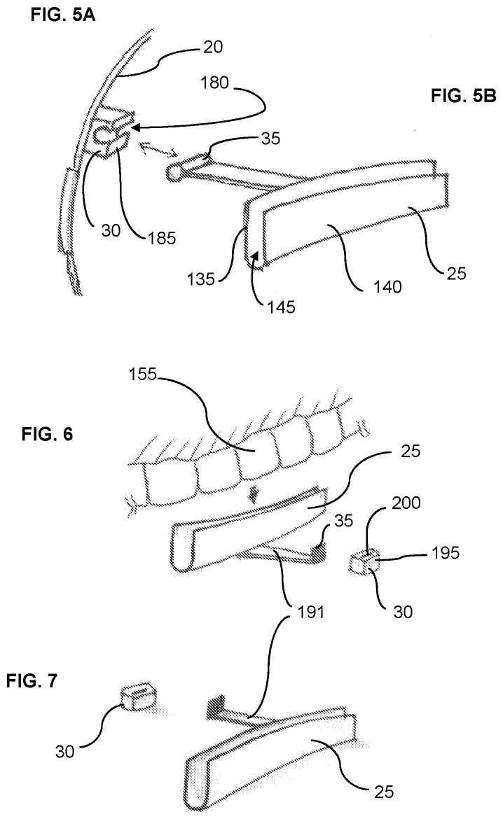

At the end (at the extremity 165) of the liprest 160 has either the receiver 30 or the insert 35 attached to the mouthpiece 25. FIG. 3B shows the mouthpiece 25 with a receiver 30 and FIGS. 5B, 6, and 7 show the mouthpiece 25 with an insert 35. The applicant believes a preferred location of the receiver 30 or the insert 35 attached to the inner surface 20 is at about the center section 170 of the inner surface 20. The size of either or both the receiver 30 and the insert 35 may vary in length for the comfort of the user when the facemask 10 is worn. One variation of the facemask 10 may have the receiver 30 or the insert 35 of the inner surface 20 be one inch or more in length. Another variation of the facemask 10 for either the receiver 30 or the insert 35 of the inner surface 20 is less than one inch in length. The length of the liprest between either the receiver 30 or the insert 35 attached and the front wall 135 should be sufficiently long to comfortably accept the upper lip 175 (shown in FIG. 11) of the user.

As shown in FIGS. 3A, 3B, 4A, and 4B, the facemask 10 may have the receiver 30 having a shape of a “C” 185 with a groove 180 and the insert 35 having a shape that tightly fits into the groove 180 of the receiver. As illustrated in the disclosure, whether the inner surface 20 has a receiver and the mouthpiece 25 has a receiver is design choice and may alternate. The insert 35 fitting into the receiver 30 with the shape of a “C” 185 (as shown in FIG. 3B) may have a shape of a “H” 190 (as shown in FIG. 3A) or have a shape of a “H” with one side that attaches to the inner surface 20 have a wider width (as shown in FIG. 4A) to have more surface area to affix to the inner surface 20.

As shown in FIGS. 5A, 5B, the inner surface 20 has a receiver 30 with a round groove 180 that mates (accommodates, receives, or accepts) with a round insert 35 at the extremity 165. In FIG. 5B, the extremity 165 is not an extension of the bottom 150 of the channel 145, and it is shown to demonstrate that such variation of the design is within the scope of this disclosure.

As shown in 6 and 7, a variation of the facemask has the receiver 30 having a shape of a rectangular box 195 with a hole 200 and the insert 35 having a shape that fits tightly into the hole 200 of the receiver 30. FIG. 6 shows a row of teeth 155 of the user about to bite in between the front wall 135 and the rear wall 140 of the mouthpiece 25.

FIGS. 8, 9, and 10 show the cutout 55 designed to cover (otherwise, expose) the nose 60 partially, substantially, all but the tip 70 of the nose 60, or none of the nose 60 except to fit snuggly below the nostrils. FIG. 8 also shows a flexible wire 205 (shown in FIG. 8) on the upper section 50 so that wire 205 may be used to better prevent air from leaking out from under the facemask. The wire 205 should have plasticity or conforming property to the contour of the face.

FIG. 11 shows a view of the user holding the mouthpiece 25 with a receiver 30 by biting into the mouthpiece 25. The upper lip 175 of the user is resting on the liprest 160 as the row of teeth 155 is inserted into the channel 145 between the front wall 135 and the rear wall 140. One advantage of the facemask presented herein is that the covering portion 210 (shown in FIG. 8), made of fabric, paper, nylon, and other flexible materials, can be replaced, while the mouthpiece can be disinfected and used over again, making the facemask 10 economical. Moreover, the upper jaw, that is the upper row of teeth 155 is fixed to the head (that is, it is the lower jaw that moves when talking or eating) the facemask 10 held by the mouthpiece 25 can be comfortably worn. For the comfort in wearing the mouthpiece 25 over an extended period of time, the mouthpiece may be made of pliable silicone or similar materials that forms to match the contour of the user. Moreover, with the left flap 75 and the right flap 85, the facemask 10 greatly reduces air escaping from around the nose, reducing the chances of spreading germs and also greatly reducing fogging of eyeglasses.

While the description, drawings, and references have been presented, shown, and described with reference to different embodiments thereof, it will be appreciated by those skilled in the art that variations in form, detail, compositions, and operation may be made without departing from the spirit and scope of the disclosure.

What is claimed is:

1. A facemask comprising an outer surface, an inner surface, a receiver, and an insert, wherein the facemask further comprises of a mouthpiece, and wherein the inner surface has either a receiver or an insert attached to the inner surface, and the mouthpiece has either the receiver or the insert attached to the mouthpiece to mate with the receiver or the insert of the inner surface.

2. The facemask of claim 1, wherein the mouthpiece has a front wall and a rear wall forming a channel with a bottom so that a user wears the facemask by inserting a row of teeth in between the front wall and the rear wall.

3. The facemask of claim 2, wherein the bottom of the channel is extended beyond the front wall of the channel to form a liprest for a lip of the user.

4. The facemask of claim 3, wherein either the receiver or the insert attached to the mouthpiece is at an extremity of the liprest.

5. The facemask of claim 4, wherein the receiver or the insert attached to the inner surface is attached to the center section of the inner surface.

6. The facemask of claim 3, wherein either the receiver or the insert of the inner surface is one inch or more in length.

7. The facemask of claim 3, wherein either the receiver or the insert of the inner surface is less than one inch in length.

8. The facemask of claim 1, wherein the receiver has a shape of a “C” having a groove, and the insert has a shape to tightly fit into the groove of the receiver.

9. The facemask of claim 1, wherein the receiver has a shape of a rectangular box having a hole, and the insert has a shape to tightly fit into the hole of the receiver.

10. The facemask of claim 1, wherein the facemask further comprises of a bridge attached to the upper section of the facemask and wherein the facemask has a cutout at the upper section to accommodate a portion of a nose of a user.

11. The facemask of claim 1, wherein the facemask further comprises of an air vent attached to a right section, a left section, a lower section, or a mouth section of the facemask.

12. The facemask of claim 10, wherein the facemask further comprises of a left flap attached to a left side of the cutout and a right flap attached to the right side of the cutout so that the left flap and the right flap cover one or more portions of the nose of the user.

13. The facemask of claim 10, wherein the bridge has a bridge-adhesive section to secure the bridge and the upper section of the facemask on the user.

14. A facemask comprising an outer surface, an inner surface, a receiver, and an insert, wherein the facemask further comprises of a mouthpiece, and wherein the inner surface has a receiver attached to the inner surface, and the mouthpiece has an insert attached to the mouthpiece to mate with the receiver of the inner surface.

15. The facemask of claim 15, wherein the mouthpiece has a front wall and a rear wall, forming a channel with a bottom, so that a user wears the facemask by inserting a row of teeth in between the front wall and the rear wall, wherein the bottom of the channel is extended forming a liprest for a lip of the user, and wherein the insert attached to the mouthpiece is at an extremity of the liprest.

16. The facemask of claim 16, wherein the facemask further comprises of a bridge attached to the upper section of the facemask and wherein the facemask has a cutout at the upper section to accommodate a portion of a nose of a user.

17. The facemask of claim 16, wherein the receiver has a shape of a “C” having a groove, and the insert has a shape to tightly fit into the groove of the receiver.

18. A facemask comprising an outer surface, an inner surface, a receiver, and an insert, wherein the facemask further comprises of a mouthpiece, and wherein the inner surface has an insert attached to the inner surface, and the mouthpiece has a receiver attached to the mouthpiece to mate with the receiver of the inner surface.

19. The facemask of claim 18, wherein the mouthpiece has a front wall and a rear wall forming a channel with a bottom so that a user wears the facemask by inserting a row of teeth in between the front wall and the rear wall, wherein the bottom of the channel is extended forming a liprest for a lip of the user, and wherein the receiver attached to the mouthpiece is at an extremity of the liprest.

20. The facemask of claim 19, wherein the receiver has a shape of a rectangular box having a hole and the insert has a shape to tightly fit into the hole of the receiver, wherein the facemask further comprises of a bridge attached to the upper section of the facemask, wherein the facemask has a cutout at the upper section to accommodate a portion of a nose of a user, wherein the bridge has a bridge-adhesive section to secure the bridge and the upper section of the facemask on the user, and wherein the left section has a left-adhesive section and the section has a right-adhesive section to secure the facemask to the user.
